(19) United States
(12) Patent Application Publication
(10) Pub. No.: US 2017/0065239 A1
(43) Pub. Date: Mar. 9, 2017

(54) X-RAY CT APPARATUS AND MEDICAL IMAGE DISPLAY METHOD

(71) Applicant: HITACHI, LTD., Tokyo (JP)

(72) Inventor: Takanori HIGUMA, Tokyo (JP)

(21) Appl. No.: 15/122,703

(22) PCT Filed: Feb. 18, 2015
(86) PCT No.: PCT/JP2015/054381

§ 371 Date: Aug. 31, 2016

(30) Foreign Application Priority Data
Mar. 11, 2014 (JP) 2014-047583

(51) Int. Cl. A61B 6/00; A61B 6/06; A61B 6/03

(52) U.S. Cl. CPC A61B 6/465; A61B 6/032; A61B 6/54; A61B 6/469; A61B 6/4435; A61B 6/06; A61B 6/468; A61B 6/484

(57) ABSTRACT

In order so that a scan range or a reconstruction range can be efficiently confirmed within a limited time during examination, the present invention relates to an X-ray CT apparatus that acquires and displays tomographic images of an object and is characterized by comprising a mode setting unit that sets a display time per confirmation image or the number of the confirmation images to be displayed as a mode that displays the confirmation images to be used for confirming the scan range and the reconstruction range according to a predetermined wait time and a display control unit that displays the confirmation images according to the display time or the number of display images set by the mode setting unit.

### TECHNICAL FIELD

The present invention relates to an X-ray CT (Computed Tomography) apparatus and a technique for efficiently confirming a scan range and a reconstruction range within a limited time during examination.

### BACKGROUND ART

An X-ray CT apparatus is an apparatus that reconstructs tomographic images based on projection data from various angles acquired by irradiating an X-ray from the vicinity of an object and displays the tomographic images. In a recent X-ray CT apparatus, the scanner speed and the number of rows of the X-ray detector have been increased, which can acquire a large amount of detailed images in a short time. On the other hand, it is desired to improve the examination throughput because a large amount of images are generated.

In order to improve the examination throughput, Patent Literature 1 discloses that an image referred to as a real-time reconstruction image or a preview image, in which the entire image was roughly constructed, is displayed during scanning or after scanning and is used for confirming a scan range and an injection state of contrast medium, specifying a range to reconstruct a detailed image, and the like.

### CITATION LIST

### Patent Literature

PTL 1: Japanese Unexamined Patent Publication No. 9-294739

### SUMMARY OF INVENTION

### Technical Problem

However, in PTL 1, no consideration is made to efficiently confirm a scan range and a reconstruction range within a limited time during examination. For example, in a contrast examination, a scan starts after contrast medium reaches a predetermined site, and there is time to wait for the contrast medium reaching during the examination. More specifically, in a case of performing contrast examination for the liver, scans are performed in each phase referred to as an artery phase, a portal vein phase, and an equilibrium phase, and wait times are generated between the artery phase and the portal vein phase and between the portal vein phase and the equilibrium phase. By using such wait times for confirming the scan range and the reconstruction range, a need to improve the examination throughput is generated.

Therefore, the present invention has a purpose to provide an X-ray CT apparatus that can efficiently confirm a scan range or a reconstruction range within a limited time during examination.

### Solution to Problem

In order to achieve the above purpose, the present invention is characterized by setting a mode that displays confirmation images to be used for confirming a scan range and a reconstruction range according to a limited wait time during examination and displaying the confirmation images according to the set mode.

Specifically, the present invention is an X-ray CT apparatus that acquires and displays a tomographic image of an object and, as a mode that displays confirmation images to be used for confirming a scan range or a reconstruction range according to a predetermined wait time, is characterized by comprising a mode setting unit that sets a display time per confirmation image or the number of the confirmation images to be displayed and a display control unit that displays the confirmation images according to the display time or the number of images set by the mode setting unit.

### Advantageous Effects of Invention

According to the present invention, an X-ray CT apparatus that can efficiently confirm a scan range and a reconstruction range within a limited time during examination can be provided.

### BRIEF DESCRIPTION OF DRAWINGS

FIG. 1 is an overall configuration diagram of an X-ray CT apparatus of the present invention.

FIG. 2 is a diagram showing an example process flow of the contrast examination.

FIG. 3 is a diagram showing a scan range to be specified on a scanogram image.

FIG. 4 is a functional block diagram of a first embodiment.

FIG. 5 is a diagram showing a process flow of the first embodiment.

FIG. 6 is a diagram showing an example of a display mode input window.

FIG. 7 is a diagram showing display directions of images.

FIG. 8 is a diagram explaining an example of the alternate display.

FIG. 9 is a diagram explaining a modification of the alternate display.

FIG. 10 is a diagram showing an example of a necessity input window.

FIG. 11 is a diagram showing the other example of the necessity input window.

FIG. 12 is a diagram explaining an order to display images in a second embodiment.

FIG. 13 is a diagram showing an example of the necessity input window of the second embodiment.

FIG. 14 is a diagram explaining an order to display images in a third embodiment.

FIG. 15 is a diagram showing an example of a multiple-condition setting input window.

### DESCRIPTION OF EMBODIMENTS

Hereinafter, a desirable embodiment of the X-ray CT apparatus related to the present invention will be described according to the attached drawings. Additionally, in the following description and the attached drawings, repeated descriptions for components having the same functional configuration will be omitted by assigning the same reference sign.

### First Embodiment

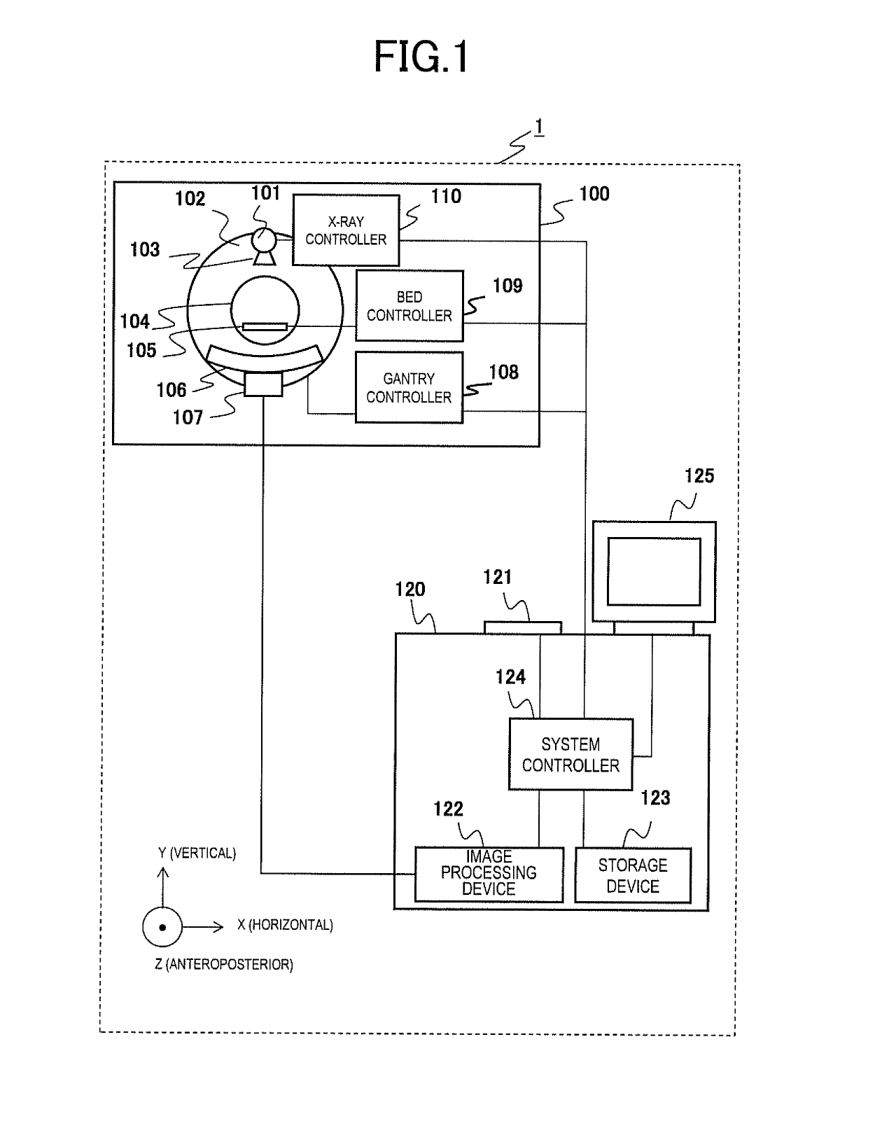

FIG. 1 is a block diagram showing the overall configuration of an X-ray CT apparatus. As shown in FIG. 1, an X-ray CT apparatus 1 comprises a scan gantry unit 100 and an operation unit 120.

The scan gantry unit 100 comprises an X-ray tube device 101, a rotary disk 102, a collimator 103, an X-ray detector 106, a data acquisition system 107, a bed device 105, a gantry controller 108, a bed controller 109, and an X-ray controller 110.

The X-ray tube device 101 is a device that irradiates an X-ray to an object placed on the bed device 105. The collimator 103 is a device that limits a radiation range of the X-ray to be irradiated from the X-ray tube device 101. The rotary disk 102 is provided with an opening 104 to accommodate the object placed on the bed device 105, is equipped with the X-ray tube device 101 and the X-ray detector 106, and rotates around the object. The X-ray detector 106 is a device that is disposed opposite to the X-ray tube device 101 and measures a spatial distribution of transmitted X-rays by detecting X-rays transmitted through the object, in which a number of detection elements are one-dimensionally arranged in the rotational direction of the rotary disk 102 or a number of detection elements are two-dimensionally arranged in the rotational direction and the rotation-axis direction of the rotary disk 102.

The data acquisition system 107 is a system to acquire an X-ray amount detected by the X-ray detector 106 as digital data. The gantry controller 108 is a device to control rotation and gradient of the rotary disk 102. The bed controller 109 is a device to control the up-down, back-forth, and left-right movements of the bed device 105. Additionally, the respective up-down, back-forth, and left-right directions are as shown in FIG. 1 and are also referred to as the Y, Z, and X directions respectively in the subsequent description. The X-ray controller 110 is a device to control the electric power to be input to the X-ray tube device 101.

The operation unit 120 comprises an input device 121, an image processing device 122, a display device 125, a storage device 123, and a system controller 124. The input device 121 is a device to input an object name, an examination date, a scanning condition, and the like, and, specifically, is a keyboard, a pointing device, a touch panel, or the like.

The image processing device 122 is a device that performs calculation processing for measurement data to be exported from the data acquisition system 107 to reconstruct a CT image. The display device 125 is a device to display the CT image and the like generated by the image processing device 122 and, specifically, is a CRT (Cathode-Ray Tube), a liquid crystal display, or the like.

The storage device 123 is a device to store data acquired by the data acquisition system 107, CT image data generated by the image processing device 122, and the like and, specifically, is an HOD (Hard Disk Drive) or the like. The system controller 124 is a device to control these devices, the gantry controller 108, the bed controller 109, and the X-ray controller 110. Also, the system controller 124 may execute a process flow to be described later.

The X-ray tube device 101 irradiates an X-ray to an object according to scanning conditions by that the X-ray controller 110 controls the electric power to be input to the X-ray tube device 101 based on the scanning conditions such as an X-ray tube voltage and an X-ray tube current in particular that were input from the input device 121. The X-ray detector 106 uses a number of X-ray detection elements to detect X-rays irradiated from the X-ray tube device 101 and transmitted through the object and measures a transmitted X-ray distribution. The rotary disk 102 is controlled by the gantry controller 108 and rotates based on a scanning condition such as a rotational speed in particular that was input from the input device 121. The bed device 105 is controlled by the bed controller 109 and operates based on a scanning condition such as a helical pitch in particular that was input from the input device 121.

Measuring a transmitted X-ray distribution by X-ray irradiation from the X-ray tube device 101 and the X-ray detector 106 is repeated with rotation of the rotary disk 102, which can acquire projection data from various angles. In the projection data, views that represent the respective angles, channel (ch) numbers that are detection element numbers of the X-ray detector 106, and row numbers are associated with each other. The projection data acquired from various angles is transmitted to the image processing device 122. The image processing device 122 performs a back projection process for the transmitted projection data from various angles to reconstruct a CT image. The CT image acquired by the reconstruction is displayed on the display device 125.

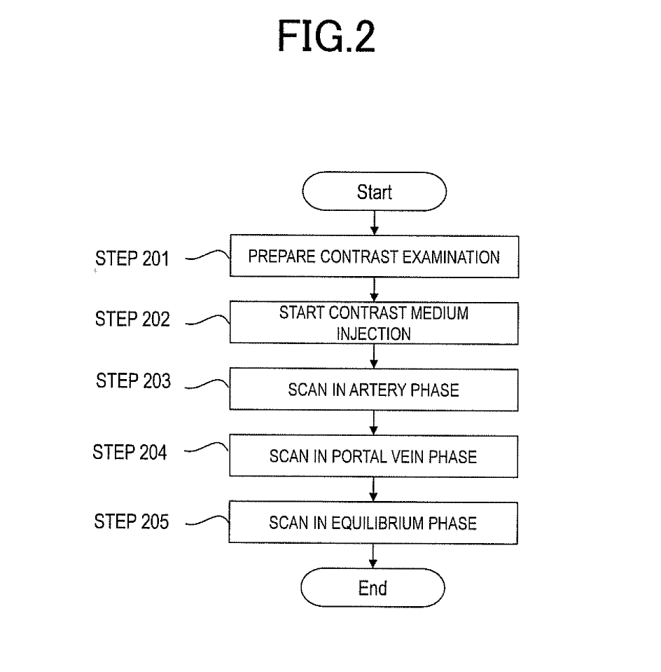

Using FIG. 2, a contrast examination of a liver will be described as an example of the examination flow to which the present embodiment is applied. Hereinafter, each step will be described.

(Step 201)

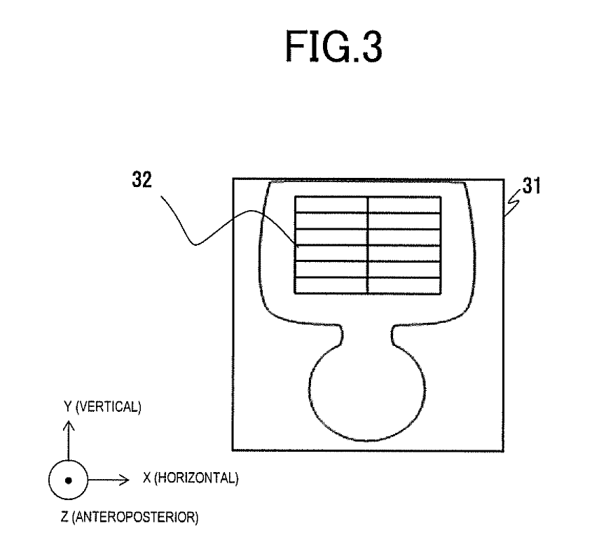

An operator prepares a contrast examination. Specifically, the operator confirms object information, guides the object to the bed device 105, and then inputs contrast examination conditions through the input device 121. When inputting the contrast examination conditions, a scan range or a reconstruction range, i.e. a length along the Z direction that is the body-axis direction of the object is set on a scanogram image 31 shown in FIG. 3. In order to set the scan range or the reconstruction range, for example, a frame 32 for setting a range is used as shown in FIG. 3, and a position and a size of the frame 32 are set through the input device 121. Additionally, the line parallel to the Z direction in the frame 32 shows the center of a tomographic image, and a plurality of lines orthogonal to the Z direction show positions in the body-axis direction of the tomographic image.

(Step 202)

Injecting contrast medium into the object starts. The contrast medium injected into the object reaches the liver after circulating through the body. After the contrast medium reaches the liver, scans in the subsequent steps are executed.

(Step 203)

A scan in an artery phase is executed. The scan in the artery phase is executed in approximately 40 seconds after the contrast medium was injected.

(Step 204)

A scan in a portal vein phase is executed. The scan in the portal vein phase is executed in approximately 60 seconds after the contrast medium was injected.

(Step 205)

A scan in an equilibrium phase is executed. The scan in the equilibrium phase is executed in approximately 180 seconds after the contrast medium was injected.

Between each scan to be performed in Steps 203 to 205, a wait time of approximately 20 to 120 seconds is generated. In the present embodiment, acquired images are efficiently displayed during such a wait time to allow an operator to confirm the images, which improves an examination throughput. In particular, this is helpful to confirm whether or not a scan range or a reconstruction range set in Step 201 is appropriate.

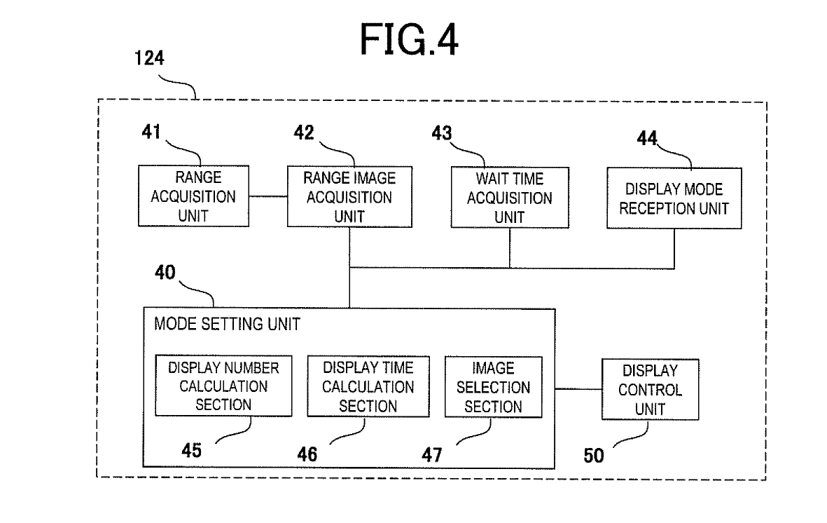

Using FIG. 4, essential parts of the present embodiment will be described. Additionally, these essential parts may be configured by exclusive hardware or may be configured by software operating on the system controller 124. Here, a case of the configuration by software will be described.

As a mode that displays confirmation images to be used for confirming a scan range or a reconstruction range according to a predetermined wait time, the present embodiment comprises a mode setting unit 40 that sets a display time per confirmation image or the number of the confirmation images to be displayed and a display control unit 50 that displays the confirmation images according to the display time or the number of images set by the mode setting unit 40.

Also, as components supporting operations of the mode setting unit 40, the mode setting unit 40 comprises a range acquisition unit 41, a range image acquisition unit 42, a wait time acquisition unit 43, and a display mode reception unit 44. Hereinafter, each component will be described.

The range acquisition unit 41 acquires a predetermined scan range or a predetermined reconstruction range. The scan range or the reconstruction range to be acquired may be a range input through the input device 121, for example, the frame 32 set on a scanogram image or a range stored in the storage device 123.

The range image acquisition unit 42 acquires tomographic images in the scan range or the reconstruction range acquired by the range acquisition unit 41. All or an arbitrary number of the tomographic images acquired by the range image acquisition unit 42 are treated as confirmation images to be used for confirming the scan range and the reconstruction range.

The wait time acquisition unit 43 acquires a wait time to be generated during examination. The wait time to be acquired may be a time calculated based on contrast examination conditions, for example, a differential value between scan start times of the respective phases during a contrast examination of a liver or may be a time preset according to the examination conditions. During the acquired wait time, confirmation images are displayed.

The display mode reception unit 44 receives information to be used for setting a mode that displays confirmation images from, for example, an input to a display mode input window to be described later. The information to be input includes, for example, the number of display images, a display time per image, an image display order, a type of display image, a specified range, and the like. Each of the information to be received by the display mode reception unit 44 will be described later with the display mode input window.

The mode setting unit 40 sets a display time per confirmation image or the number of the confirmation images to be displayed as a mode that displays the confirmation images according to the predetermined wait time. The mode setting unit 40 comprises at least either one of a display number calculation section 45 or a display time calculation section 46 and an image selection section 47. Hereinafter, each section will be described.

The display number calculation section 45 calculates the number of confirmation images to be displayed within a wait time acquired by the wait time acquisition unit 43. For example in a case where it is set to display all the tomographic images in a scan range or a reconstruction range, the number of the tomographic images in the scan range or the reconstruction range are calculated as the number of confirmation images to be displayed. Also, in a case where a display time per image is set, the number of the confirmation images to be displayed may be calculated by the following formula.

Nd=Tw/T1 <Formula 1>

In the above formula, Nd is the number of confirmation images to be displayed, Tw is a wait time, and T1 is a display time per image.

The display time calculation section 46 calculates a time to display each confirmation image. For example, in a case where the number of confirmation images to be displayed is set, a display time per confirmation image may be calculated by the following formula.

T1=Tw/Nd <Formula 2>

In the above formula, T1 is a display time per image, Tw is a wait time, and Nd is the number of confirmation images to be displayed.

The image selection section 47 selects images treated by the display control unit 50, i.e. confirmation images to be displayed on the display device 125 from a scan range or a reconstruction range. An order of the confirmation images to be selected from the scan range or the reconstruction range is set according to a mode. For example, each of or each plurality of the confirmation images may be selected in order from one end position toward the other end position in the scan range or the reconstruction range, or the confirmation images may be selected in an alternate order from both of the end positions of the scan range or the reconstruction range toward the center position.

Also, while each plurality of images is being selected in one direction, it may be set so as to switch the selected direction to the inverse direction.

The display control unit 50 displays confirmation images on the display device 125 according to a mode set by the mode setting unit 40. Display modes will be described later.

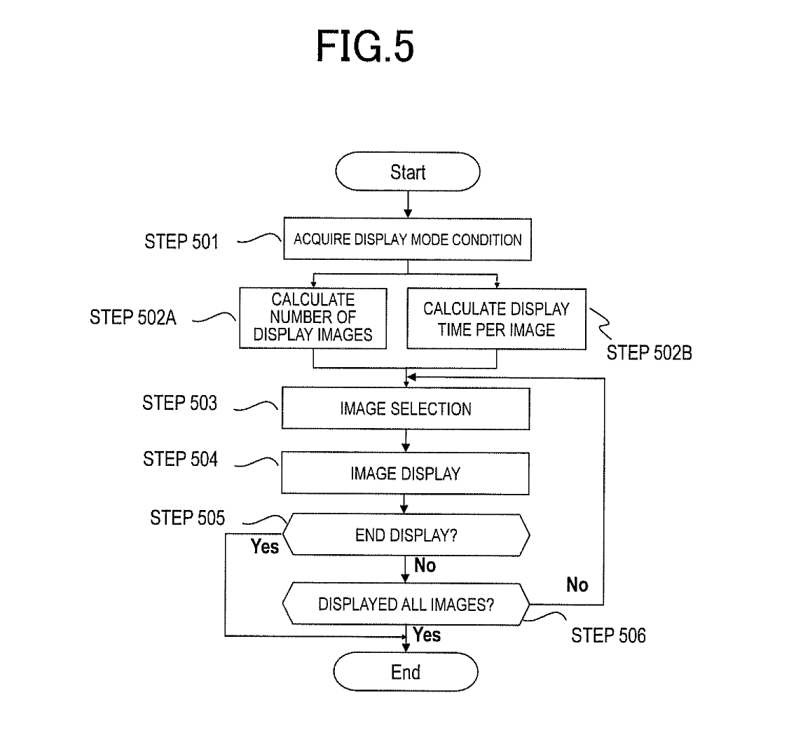

Using FIG. 5, the flow of image display processes to be executed by the X-ray CT apparatus 1 comprising the above components will be described.

(Step 501)

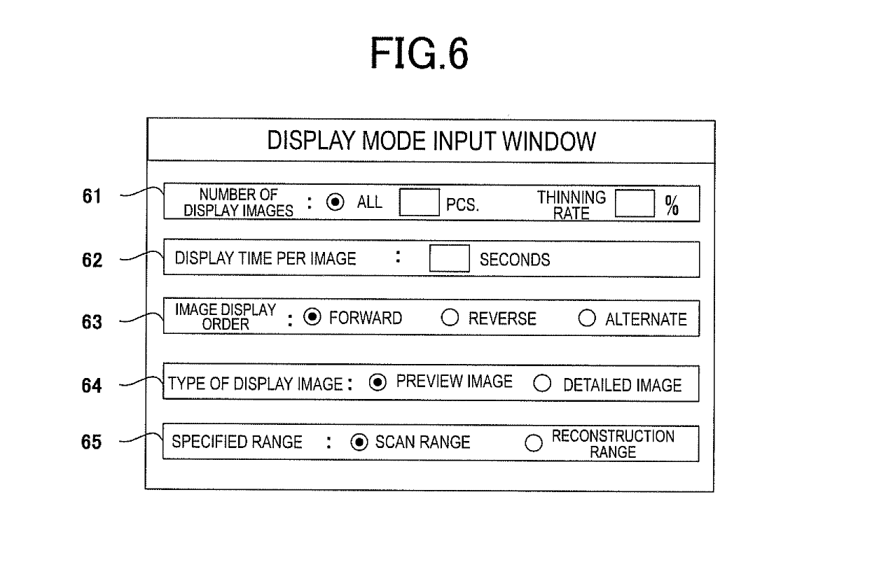

The display mode reception unit 44 receives display mode conditions. In order to input the display mode conditions, for example, a display mode input window as shown in FIG. 6 is used. The display mode input window of FIG. 6 includes an image number input field 61, a time input field 62, a display order input field 63, an image type input field 64, and a specified range input field 65. Hereinafter, each input field will be described with information to be input.

The image number input field 61 is an input field to input the number of confirmation images to be displayed during a wait time. The image number input field 61 of FIG. 6 includes a radio button to specify the number of all images in scan ranges or reconstruction ranges, a text box to input the specific number of images, and a text box to specify a thinning rate in order from the left. In order to confirm all the tomographic images in the scan range or the reconstruction range, an operator can select radio buttons as shown in FIG. 6.

In order to specify the specific number of images, a numerical value is input in the text box to specify the number of images. Additionally, the number of images to be input in the image number input field 61 may also be the upper limit number of confirmation images to be displayed during a wait time. Also, in order to thin out the number of the confirmation images, a thinning rate is input in the text box to specify the thinning rate. The thinning rate is a real number equal to or more than 0 and less than 100, and the confirmation images are not thinned out when 0 is input as the thinning rate, which results in specifying all of scan ranges or reconstruction ranges as the number of the confirmation images.

The time input field 62 is an input field to input a display time per confirmation image. In the time input field 62 of FIG. 6, a text box to input a display time per image is disposed. An operator can input a time required to determine necessity of the confirmation images in the text box.

The display order input field 63 is an input field to input an order for displaying confirmation images in a scan range or a reconstruction range. In the display order input field 63 of FIG. 6, the radio buttons to specify each of forward display, reverse display, and alternate display are arranged in order from the left, and the forward display radio button is specified.

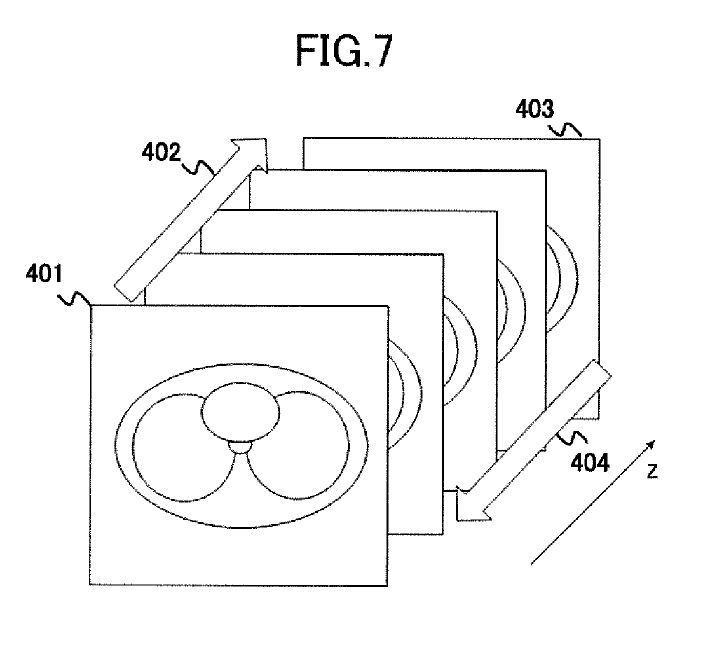

Using FIG. 7, the forward display and the reverse display will be described. Confirmation images in a scan range or a reconstruction range are arranged along the body-axis direction of an object, i.e. the Z direction from a start image 401 that is a confirmation image in a scan start position to an end image 403 that is a confirmation image in a scan end position in each position in the Z direction. The forward display is used when the confirmation images are displayed in the same direction as the Z direction, i.e. when the confirmation images are displayed from the start image 401 toward the end image 403, and the direction is the same as an arrow 402 in FIG. 7. The reverse display is used when the confirmation images are displayed in a reverse direction to the forward direction, and the direction is the same as an arrow 404 of FIG. 7. When the forward display or the reverse display is used for displaying the confirmation images, the confirmation images can be confirmed while considering sequence of the organs in body-axis direction sequence in the images, which makes easy for an operator to anatomically determine necessity. Also, the forward display may be referred to as the reverse display inversely.

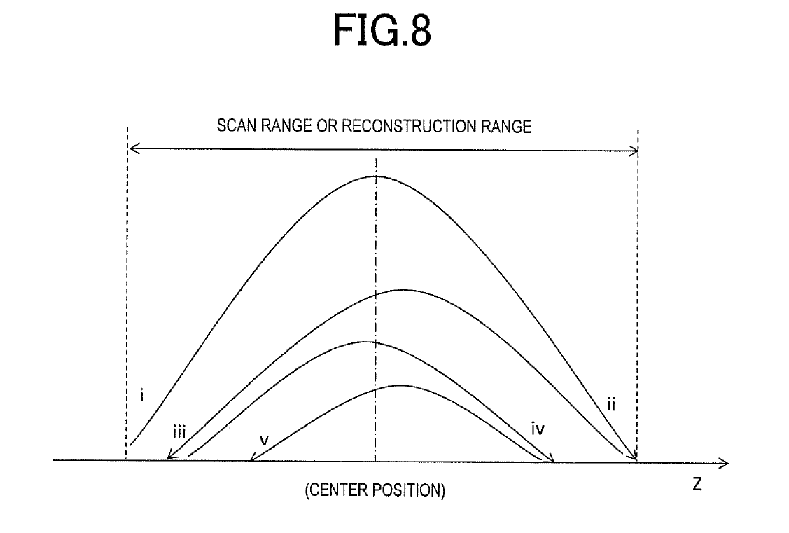

Using FIG. 8, an example of the alternate display will be described. In FIG. 8, the horizontal axis shows the Z direction, i.e. positions in the body-axis direction, and an order to display confirmation images is indicated by circled numerals. The alternate display of FIG. 8 starts displaying the confirmation images on both the ends alternately by shifting a position from an end toward the center, i.e. from either one of both the ends in a scan range or a reconstruction range (i) to the other end position (ii), a position shifted closer to the center than one end (iii), a position shifted further closer to the center than the other end (iv), and a position shifted further closer to the center than the one end (v).

In most cases, a region of interest is in the center position of a scan range or a reconstruction range, and unnecessary tomographic images are generated in the vicinity of the end positions of the scan range or the reconstruction range. By performing the alternate display of FIG. 8, displaying confirmation images that are likely to be unnecessary is prioritized, which can shorten a time required to determine necessity for the confirmation images.

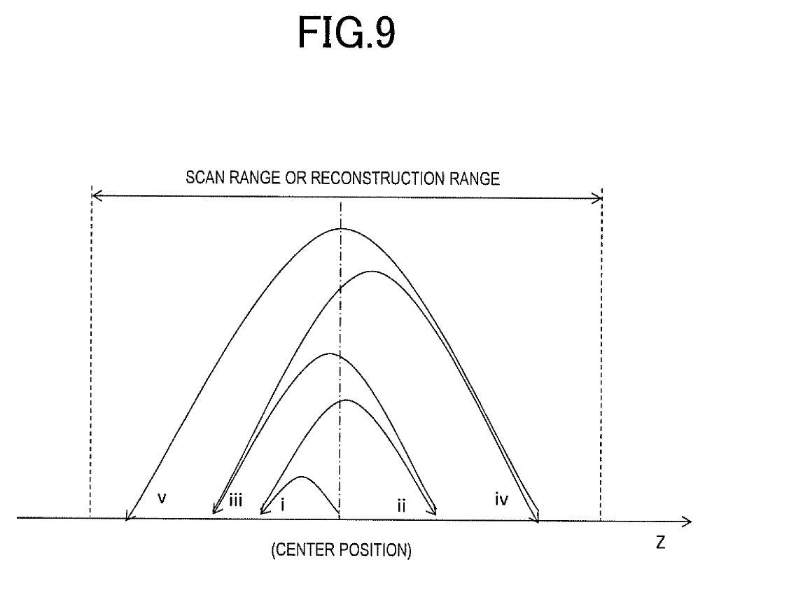

Using FIG. 9, a modification of the alternate display will be described. In FIG. 9, the horizontal axis shows the Z direction, i.e. positions in the body-axis direction, and an order to display confirmation images is indicated by circled numerals. The alternate display of FIG. 9 starts displaying the confirmation images on both the ends alternately by shifting a position from the center toward the ends, i.e. from the center position in a scan range or a reconstruction range (i) to a position shifted from the center to either one of both the ends (ii), a position shifted from the center to the other of both the ends (iii), a position further shifted from the center to the one end (iv), and a position further shifted from the center to the other end (v). The alternate display of FIG. 9 can shorten a time required to determine necessity for the confirmation images when an amount of unnecessary images in the scan range or the reconstruction range are larger than that of necessary images.

The image type input field 64 is an input field to input a type of images to be displayed as confirmation images. The radio buttons to specify a preview image and a detailed image are arranged in order from the left in the image type input field 64 of FIG. 6, and the radio button for the preview image is specified.

A preview image is a tomographic image reconstructed in a shorter time than the scan time, and the entire image is roughly constructed. The preview image can also be referred to as a real-time reconstruction image. A detailed image is a tomographic image generated in normal reconstruction, and in most cases, although it takes a longer time to perform reconstruction processing than the scan time, the image is appropriate to check the details. When an operator confirms a tomographic image scanned immediately before, the preview image should be specified. Also, when image details need to be checked and sufficient time has elapsed after the scan, the detailed image should be specified in a case, for example, where there are some tomographic images for which normal reconstruction processing has been performed.

The specified range input field 65 is an input field to input whether or not a range to be set by confirming confirmation images is a scan range or a reconstruction range. The radio buttons to specify a scan range or a reconstruction range are arranged in order from the left in the specified range input field 65 of FIG. 6, and the radio button for the scan range is specified. An operator can select either of the radio buttons according to a purpose to confirm.

Additionally, FIG. 6 is an example, and a display mode input window is not limited to the configuration of FIG. 6.

The display mode input window should include an input field at least equivalent to either one of the image number input field 61 and the time input field 62.

When display mode conditions are acquired in Step 501, the procedure proceeds to either of Step 502A or Step 502B.

(Step 502A)

The display number calculation section 45 calculates the number of confirmation images to be displayed within a wait time. In order to calculate the number, for example, <Formula 1> may be used. When using <Formula 1>, a wait time acquired by the wait time acquisition unit 43 is used as Tw, and a display time per image input in the time input field 62 is used as T1. Additionally, the T1 value may be a time set according to the examination conditions previously stored in the storage device 123.

(Step 502B)

The display time calculation section 46 calculates a time to display each confirmation image. In order to calculate the time, for example, <Formula 2> may be used. When using <Formula 2>, a wait time acquired by the wait time acquisition unit 43 is used as Tw, and the number of the confirmation images input in the image number input field 61 is used as Nd.

Additionally, when a thinning rate is input in the image number input field 61, the number of confirmation images to be displayed is calculated by the following formula.

Nd=(100−Rt)·Nt/100 <Formula 3>

In the above formula, Nd is the number of confirmation images to be displayed, Rt is a thinning rate (%), and Nt is the total number of tomographic images in a scan range or a reconstruction range.

(Step 503)

The image selection section 47 selects a confirmation image to be displayed on the display device 125 from among a scan range or a reconstruction range. The confirmation images are selected according to the number of the confirmation images input from the image number input field 61 or the number of the confirmation images calculated by the display number calculation section 45 and an order to display the confirmation images input from the display order input field 63. Hereinafter, a couple of examples will be described.

When the number of confirmation images is all and a display order is forward display or reverse display, each of the confirmation images is selected by shifting a position from one end toward the other end in a scan range or a reconstruction range.

When the number of confirmation images is all and a display order is alternate display, each of the confirmation images on both the ends is alternately selected by shifting a position from one end toward the center in a scan range or a reconstruction range. Alternatively, each of the confirmation images on both the ends is alternately selected by shifting a position from the center toward an end.

When a display order is forward display or reverse display and the number of confirmation images is not all, for example, in a case where a thinning rate is input in the image number input field 61, the confirmation images are selected by skipping the number of the confirmation images according to the thinning rate from one end position toward the other end position. The number of the confirmation images to be skipped is calculated by the following formula.

Ns=Rt/(100−Rt) <Formula 4>

In the above formula, Ns is the number of images to be skipped, and Rt is a thinning rate (%).

For example, according to <Formula 4>, images are skipped every other image in a case where the thinning rate is 50%; every two images in a case where the thinning rate is 67%; and every nine images in a case where the thinning rate is 90%.

When a display order is alternate display and a thinning rate is input, positions in the Z direction are first set based on the number of images to be skipped that is calculated according to the thinning rate, and confirmation images on both the ends of the set positions are selected alternately.

(Step 504)

The display control unit 50 displays confirmation images selected by the image selection section 47 on the display device 125. A display time per image calculated in Step 502B or that acquired through the time input field 62 in Step 501 is set as a display time of a confirmation image. After the confirmation image is displayed, the procedure proceeds to Step 505 when the set display time elapses. By thus setting the display time, all the confirmation images can be displayed during a wait time. Also, the next phase scan does not start before confirming all the confirmation images finishes.

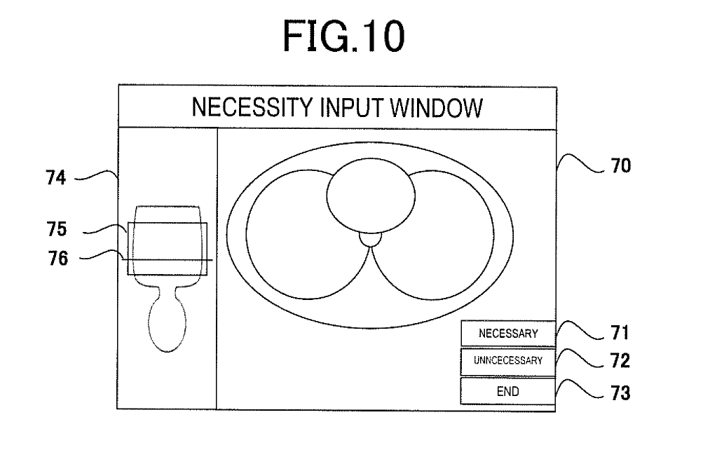

An operator confirms confirmation images displayed on the display device 125 to determine whether or not the said image position is necessary. A necessity determination result may be input in the necessity input window as shown in FIG. 10. The necessity input window in FIG. 10 will be described. The necessity input window in FIG. 10 includes a confirmation image display portion 70 and a scanogram image display portion 74. Hereinafter, each portion will be described.

The confirmation image display portion 70 is a display portion on which confirmation images selected by the image selection section 47 in Step 503 are displayed. In the confirmation image display portion 70, a necessary button 71, an unnecessary button 72, and an end button 73 that are clickable with a mouse cursor are disposed. The necessary button 71 is a button to input that positions of the displayed confirmation images are necessary. The unnecessary button 72 is a button to input that positions of the displayed confirmation images are unnecessary.

The end button 73 is a button for instructing to end display of confirmation images. An operator determines whether or not positions of the displayed confirmation images are necessary or unnecessary in scans and reconstruction processes in the subsequent phases by confirming the displayed confirmation images on the confirmation image display portion 70 and clicks any one of the necessary button 71, the unnecessary button 72, and the end button 73 according to the determination result. In a case where any one of the three buttons is clicked, the procedure may proceed to Step 505 without waiting for elapse of the set display time. Also, in a case where none of the buttons are clicked when the set display time elapsed, it may be set so as to determine that the positions of the displayed confirmation images are necessary.

The scanogram image display portion 73 is a display portion on which a scanogram image is displayed. A frame 74 and a line 75 are displayed on the scanogram image to be displayed on the scanogram image display portion 73.

The frame 74 is a graphic to indicate a preset scan range or a reconstruction range.

The line 75 is a graphic to indicate a position of confirmation image displayed on the confirmation image display portion 70. The line 75 position on a scanogram image moves when the confirmation images are switched. An operator can understand a position of the displayed confirmation image by checking positions of the frame 74 and the line 75 displayed on the scanogram image. Additionally, instead of the scanogram image display portion 73, it may be configured so as to display numeric values that shows a confirmation image position.

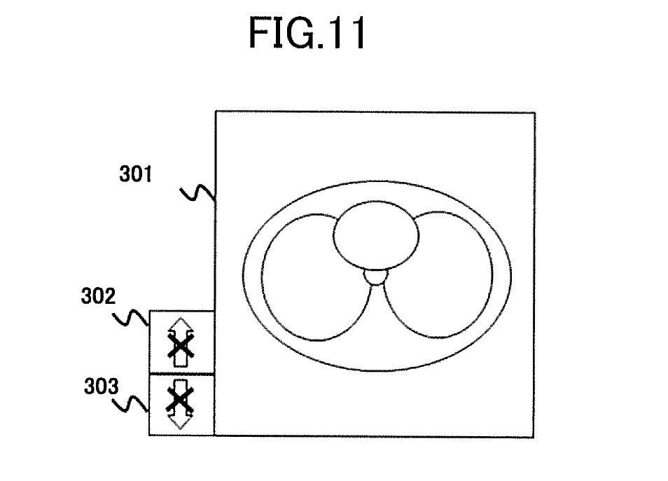

Using FIG. 11, the other example of a necessity input window will be described. The necessity input window of FIG. 10 is a window to input necessity determination for each of confirmation images to be displayed. The necessity input window of FIG. 11 includes an unnecessary herebefore button 302 and an unnecessary hereafter button 303 in addition to a confirmation image display portion 301. The unnecessary herebefore button 302 is a button to input that confirmation images displayed before the currently displayed image are unnecessary. The unnecessary hereafter button 303 is a button to input that confirmation images to be displayed after the currently displayed image are unnecessary.

An operator can click the unnecessary herebefore button 302 at a position where necessity determination changes from unnecessity to necessity while forward-direction display or reverse-direction display is being made. Then, the operator can click the unnecessary hereafter button 303 at a position where the necessity determination changes from necessity to unnecessity. Also, it may be set so as to instruct to end the display when the unnecessary hereafter button 303 is clicked. Furthermore, the scanogram image display portion 73 shown in FIG. 10 may be disposed in the necessity input window of FIG. 11.

(Step 505)

The display control unit 50 determines whether or not an instruction to end display of confirmation images was made. For example, this is determined by whether or not the end button 73 is clicked in the necessity input window of FIG. 10. In a case where the determination result is “Yes”, i.e. in a case where ending the display is instructed, the image display processes in FIG. 5 end. In a case where the determination result is “No”, i.e. in a case where ending the display is not instructed, the procedure proceeds to Step 506.

(Step 506)

The display control unit 50 determines whether or not the predetermined number of confirmation images was displayed. In a case where the determination result is “Yes”, i.e. in a case where the predetermined number of confirmation images was displayed, the image display processes in FIG. 5 end. In a case where the determination result is “No”, i.e. in a case where the predetermined number of confirmation images is not displayed, the procedure goes back to Step 503.

The X-ray CT apparatus 1 executes the above flow of the image display processes, which can efficiently confirm a scan range or a reconstruction range within a limited time during examination.

### Second Embodiment

Next, a second embodiment will be described. In the first embodiment, forward display, reverse display, and alternate display are described. In the present embodiment, an order to display confirmation images is selected according to a necessity determination result. That is, in the present embodiment, operations of the image selection section 47 are different from the first embodiment, and the other configuration is similar to the first embodiment. The similar configuration will be omitted from the description.

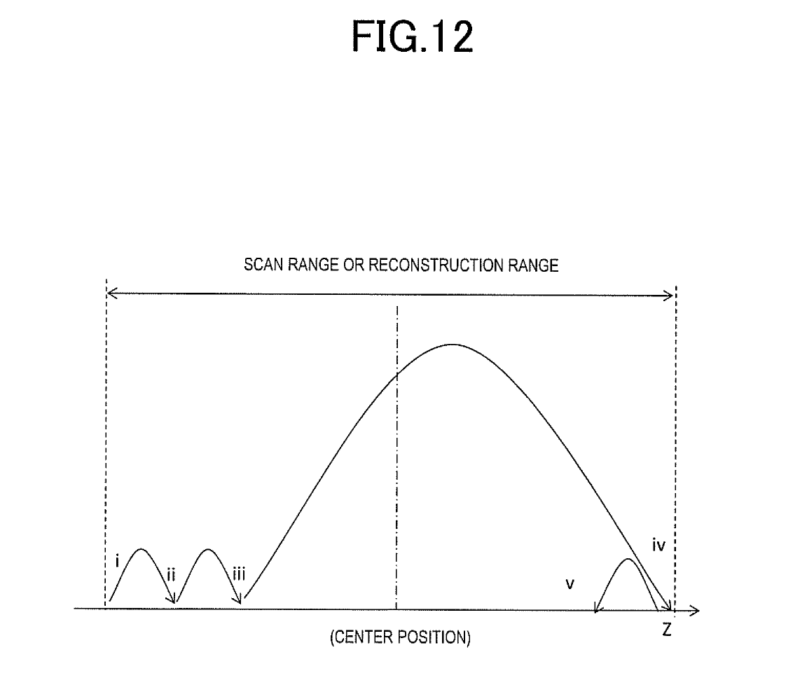

Using FIG. 12, the present embodiment will be described. In FIG. 12, the horizontal axis shows the Z direction, i.e. positions in the body-axis direction, and an order to display confirmation images is indicated by circled numerals. In the display of FIG. 12, displaying the confirmation images starts from either one (i) of both the end positions in a scan range or a reconstruction range, and the confirmation image display and necessity determination input are performed while shifting a position from one end (i) to the other end. In a case of determining to be unnecessary in each position (i and ii), a confirmation image position is shifted while every single image or the predetermined number of images is being skipped (i->ii and ii->iii).

In a case of determining to be necessary in a position (iii), the position is shifted to the other end position to display a confirmation image. After a confirmation image of the other end (iv) is displayed, displaying the confirmation images and inputting necessity determination are performed while the position is being shifted from the other end (iv) toward one end (i) (iv->v).

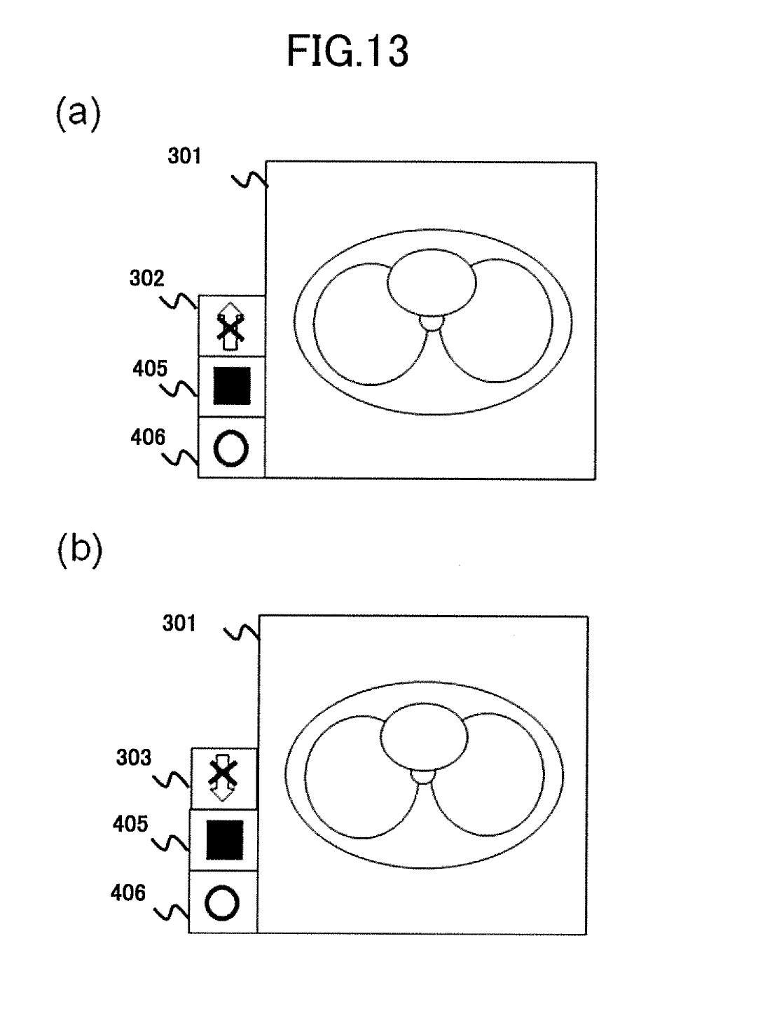

In the present embodiment, the necessity input window of FIGS. 10 and 11 may be used, or the other necessity input window may be used. The other necessity input window is described using FIG. 13. The necessity input window of FIG. 13 is a modification of FIG. 11, and the windows of FIGS. 13(a) and 13(b) are switched according to a button operation. The necessity input window of FIG. 13(a) includes the unnecessary herebefore button 302, a stop button 405, and a non-unnecessary button 406 in addition to a confirmation image display portion 301. The stop button 405 is a button to stop automatic movement of a confirmation image position and is used for switching to manual position movement. The non-unnecessary button 406 is a button to input that there are all necessary confirmation images. The necessity input window of FIG. 13(b) is the same as the necessity input window of FIG. 13(a) except including the unnecessary hereafter button 303 instead of the unnecessary herebefore button 302.

When an operator clicks the unnecessary herebefore button 302 or the non-unnecessary button 406 in the necessity input window of FIG. 13(a) while forward-direction display or reverse-direction display is being made, a confirmation image position moves to the other end position, the window is switched to the necessity input window of FIG. 13(b). Then, when the operator clicks the unnecessary hereafter button 303 or the non-unnecessary button 406 in the necessity input window of FIG. 13(b) while forward-direction display or reverse-direction display is being made, the confirmation image display ends.

In most cases, there are unnecessary confirmation images in the vicinity of the end positions in a scan range or a reconstruction range. Therefore, as shown in the present embodiment, by displaying confirmation images after shifting from a position where necessity determination is switched from unnecessity to necessity (iii) to the other end position (iv), displaying the confirmation images that are determined to be necessary can be skipped, which can shorten a time required to determine necessity for the confirmation images.

### Third Embodiment

Next, a third embodiment will be described. In the first embodiment, forward display, reverse display, and alternate display are described. In the present embodiment, an order to display confirmation images is selected according to a necessity determination result. That is, in the present embodiment, operations of the image selection section 47 are different from the first embodiment, and the other configuration is similar to the first embodiment. The similar configuration will be omitted from the description.

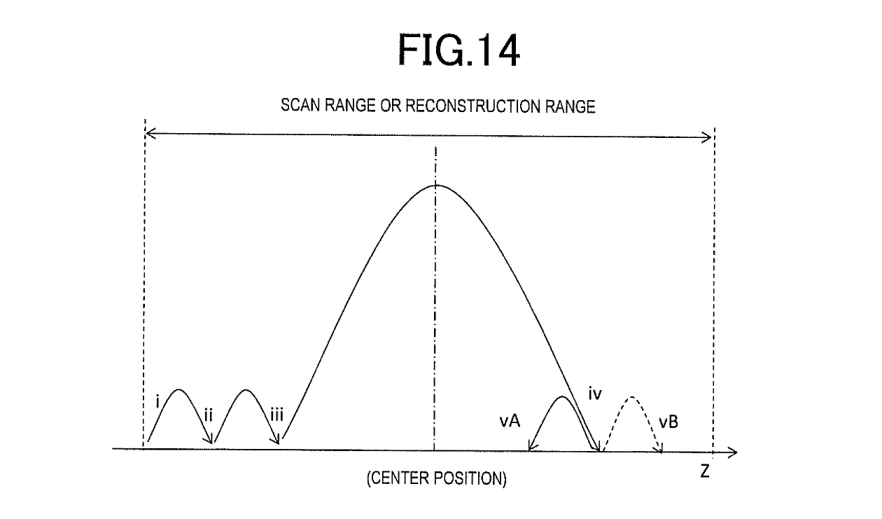

Using FIG. 14, the present embodiment will be described. In FIG. 14, the horizontal axis shows the Z direction, i.e. positions in the body-axis direction, and an order to display confirmation images is indicated by circled numerals. In the display of FIG. 14, displaying the confirmation images starts from either one (i) of both the end positions in a scan range or a reconstruction range, and the confirmation image display and necessity determination input are made while shifting a position from one end (i) to the other end. In a case of determining to be unnecessary in each position (i and ii), a confirmation image position is shifted while every single image or the predetermined number of images is being skipped (i->ii and ii->iii).

In a case of determining to be necessary in a position (iii), a confirmation image is displayed after shifting the position to a symmetrical position (iv) by taking the center position of a scan range and a reconstruction range as a reference. A distance from the said position (iii) to the center position is equal to that from the center position to the symmetrical position (iv).

In a case where a confirmation image in the symmetrical position (iv) is determined to be unnecessary, the confirmation image position is shifted toward the center position (iv->VA). In other words, a confirmation image inner than the symmetrical position (iv) is displayed. In a case where a confirmation image in the symmetrical position (iv) is determined to be necessary, the confirmation image position is shifted toward the other end position (iv->vB). In other words, a confirmation image outer than the symmetrical position (iv) is displayed.

In a case where a confirmation image in the position (vA) that was shifted from the symmetrical position (iv) to the center position is determined to be unnecessary, the confirmation image display is made while the position is being shifted toward the center position until the confirmation image is determined to be necessary. In other words, after being shifted to the symmetrical position (iv), in a case where the confirmation image is determined to be necessary while the confirmation image position is being shifted toward the center position, the confirmation image display ends.

In a case where a confirmation image in the position (vB) that was shifted from the symmetrical position (iv) to the other end position is determined to be necessary, the confirmation image display is made while the position is being shifted toward the other end position until the confirmation image is determined to be unnecessary. In other words, after being shifted to the symmetrical position (iv), in a case where the confirmation image is determined to be necessary while the confirmation image position is being shifted toward the other end position, the confirmation image display ends.

In most cases, there are necessary confirmation images in the central part in a scan range or a reconstruction range. Therefore, as shown in the present embodiment, by displaying confirmation images after shifting from a position where necessity determination is switched from unnecessity to necessity (iii) to the symmetrical position (iv), displaying the confirmation images that are determined to be necessary can be skipped, which can shorten a time required to determine necessity for the confirmation images.

Also, by automatically selecting a shifting direction (vA or vB) according to necessity determination after shifting to the symmetrical position (iv), an operator does not need to specify the shifting direction, which can save operation steps.

Furthermore, by automatically ending confirmation image display according to necessity determination while a confirmation image position is being shifted in one direction after shifting to the symmetrical position (iv), which can further shorten a time required to determine necessity for the confirmation images.

Additionally, although shifting to a symmetrical position is performed by taking the center position of a scan range or a reconstruction range as a reference in the above description, the other position may be used as a reference. For example, by taking a position previously specified by an operator as a reference, shifting to the symmetrical position may be performed.

### Fourth Embodiment

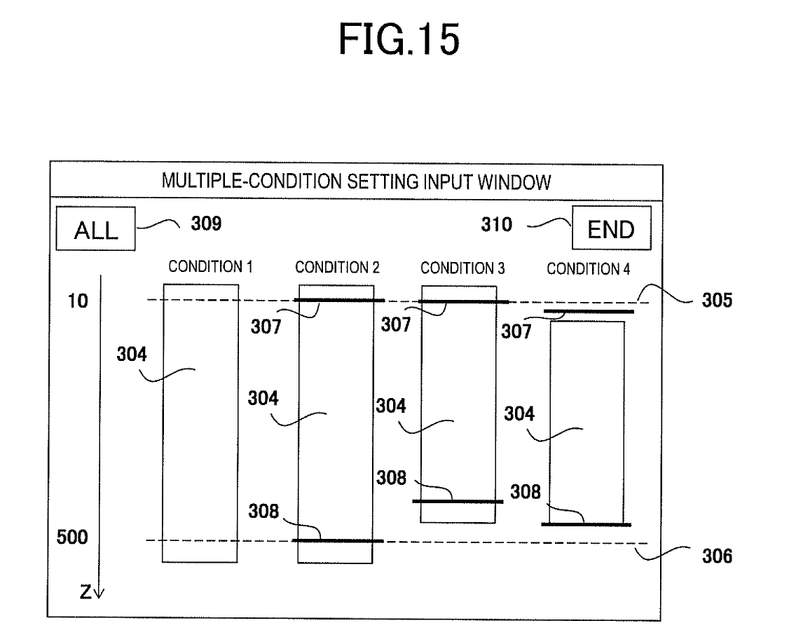

In the first to third embodiments, a scan range or a reconstruction range for one scan condition or reconstruction condition is confirmed. Depending on the examination result, there is a case where a scan range or a reconstruction range should be simultaneously confirmed for a plurality of scan conditions or reconstruction conditions. For example, such a case occurs when handling images for forwarding to a server and generating analysis images including 3D images, MPR (Multi Planer Reconstruction) images, or the like. In the present embodiment, a multiple-condition setting input window for simultaneously confirming a scan range or a reconstruction range for a plurality of conditions will be described using FIG. 15.

FIG. 15 is an example of the multiple-condition setting input window. The multiple-condition setting input window of FIG. 15 includes frames 304, a first line 305, a second line 306, first boundary specifying bars 307, second boundary specifying bars 308, an all application button 309, and an end button 310. Hereinafter, each part will be described.

The frames 304 are graphics showing a scan range or a reconstruction range set for each condition. The vertical direction shows the Z direction, i.e. the body-axis direction in the window. In FIG. 15, the same range is set for conditions 1 and 2, and a range different from the conditions 1 and 2 is set for conditions 3 and 4.

The first line 305 and the second line 306 are graphics to show a first boundary position and a second boundary position set for a condition. In FIG. 15, the line 305 showing the first boundary position in a position 10 and the line 306 showing the second boundary position in a position 500 are respectively set for the condition 1. The lines 305 and 306 have lengths that can compare the frames 304 of the conditions 2 to 4 with each other.

The first boundary specifying bars 307 and the second boundary specifying bars 308 are operating tools to be used for specifying a first boundary position and a second boundary position for each condition. An operator can specify the first boundary position and the second boundary position of each condition by moving the first boundary specifying bars 307 and the second boundary specifying bars 308 in the Z direction with a mouse operation or the like. In a case where a preset range is insufficient, as shown in the condition 4, the first boundary specifying bar 307 may be shifted outside the frame 304.

The all application button 309 is a button to apply a first boundary position and a second boundary position that were set for a condition for the other conditions. When the all application button 309 is clicked, the first boundary specifying bar 307 and the second boundary specifying bar 308 of each condition are disposed on the first line 305 and the second line 306. In FIG. 15, shown is a state where the second boundary specifying bar 308 of the condition 3 and the first boundary specifying bar 307 and the second boundary specifying bar 308 of the condition 4 were shifted after clicking the all application button 309.

The end button 310 is a button to instruct to finish setting first boundary positions and second boundary positions of all the conditions. Clicking the end button 310 finishes setting all the conditions, and the multiple-condition setting input window closes.

As described above, according to the present embodiment, a scan range or a reconstruction range can be simulataneously confirmed and can be reset for a plurality of conditions.

Additionally, the medical image display apparatus of the present invention is not limited to the above embodiments and can be embodied by modifying components without departing from the gist of the present invention. Also, a plurality of the components disclosed in the above embodiments may be combined as needed. Furthermore, several components may be deleted from all the components shown in the above embodiments.

### REFERENCE SIGNS LIST

1: X-ray CT apparatus
100: scan gantry unit
101: X-ray tube device
102: rotary disk
103: collimator
104: opening
105: bed device
106: X-ray detector
107: data acquisition system
108: gantry controller
109: bed controller
110: X-ray controller
120: operation console
121: input device
122: image calculation device
123: storage device
124: system controller
125: display device
40: mode setting unit
31: scanogram image
32: frame
41: range acquisition unit
42: range image acquisition unit
43: wait time acquisition unit
44: display mode reception unit
45: display number calculation section
46: display time calculation section
47: image selection section
50: display control unit
61: image number input field
62: time input field
63: display order input field
64: image type input field
65: specified range input field
70: confirmation image display portion
71: necessary button
72: unnecessary button
73: end button
74: scanogram image display portion
75: frame
76: line
301: confirmation image display portion
302: unnecessary herebefore button
303: unnecessary hereafter button
304: frame
305: first line
306: second line
307: first boundary specifying bar
308: second boundary specifying bar
309: all application button
310: end button
401: start image
402: arrow
403: end image
404: arrow
405: stop button
406: non-unnecessary button

1. An X-ray CT apparatus that acquires and displays tomographic images of an object, comprising:
a mode setting unit that sets a display time per confirmation image or the number of the confirmation images to be displayed as a mode that displays the confirmation images to be used for confirming a scan range and a reconstruction range according to a predetermined wait time; and
a display control unit that displays the confirmation images according to the display time or the number of display images set by the mode setting unit.

2. The X-ray CT apparatus according to claim 1, further comprising:
a necessity input portion to input whether or not the displayed confirmation image is necessary for a position of the confirmation image.

3. The X-ray CT apparatus according to claim 2,
wherein the mode setting unit has an image selection section that selects a confirmation image to be displayed next according to the input in the necessity input portion.

4. The X-ray CT apparatus according to claim 3,
wherein the image selection section starts selecting a confirmation image from one end position in the scan range or the reconstruction range and selects a confirmation image in a symmetrical position to a position where switching from unnecessity to necessity was performed by the necessity input portion in a position of the confirmation image.

5. The X-ray CT apparatus according to claim 4,
wherein the image selection section selects a confirmation image inner than the confirmation image in the symmetrical position when unnecessity is input for the confirmation image in the symmetrical position and selects a confirmation image outer than the confirmation image in the symmetrical position when necessity is input for the confirmation image in the symmetrical position.

6. The X-ray CT apparatus according to claim 3,
wherein the image selection section starts selecting a confirmation image from one end position in the scan range or the reconstruction range and selects a confirmation image in the other end position when switching from unnecessity to necessity was performed by the necessity input portion in the position of the confirmation image.

7. The X-ray CT apparatus according to claim 2,
wherein the display control unit finishes displaying the confirmation images according to the input in the necessity input portion.

8. The X-ray CT apparatus according to claim 1,
wherein the mode setting unit includes the image selection section that selects a confirmation image to be displayed next, and
the image selection section alternately selects confirmation images on one end and the other end in the scan range or the reconstruction range.

9. The X-ray CT apparatus according to claim 8,
wherein the image selection section starts selecting confirmation images from one end position in the scan range or the reconstruction range.

10. The X-ray CT apparatus according to claim 8,
wherein the image selection section starts selecting confirmation images from the center position in the scan range or the reconstruction range.

11. The X-ray CT apparatus according to claim 1,
wherein the mode setting unit sets a display time per the confirmation image according to the number of the confirmation images to be displayed within the wait time.

12. The X-ray CT apparatus according to claim 11,
wherein the number of the confirmation images is set according to a thinning rate.

13. The X-ray CT apparatus according to claim 1,
wherein the mode setting unit sets the number of the confirmation images to be displayed within the wait time according to a display time per the predetermined confirmation image.

14. The X-ray CT apparatus according to claim 1,
wherein the confirmation image is a preview image reconstructed in a shorter time than a scan time.

15. A medical image display method that acquires and displays tomographic images of an object, comprising:
a mode setting step that sets a display time per the confirmation image or the number of the confirmation images to be displayed as a mode that displays the confirmation images to be used for confirming a scan range and a reconstruction range according to a predetermined wait time; and
a display step that displays the confirmation images according to a display time or the number of display images set by the mode setting unit.
